(19) United States
(12) Patent Application Publication
(10) Pub. No.: US 2009/0138206 A1
(43) Pub. Date: May 28, 2009

(54) SYSTEM OF ANALYZING PROTEIN MODIFICATION WITH ITS BAND POSITION OF ONE-DIMENSIONAL GEL BY THE MASS SPECTRAL DATA ANALYSIS AND THE METHOD OF ANALYZING PROTEIN MODIFICATION USING THEREOF

(75) Inventors: Gun Wook Park, Daejeon-shi (KR); Kyung-Hoon Kwon, Daejeon-shi (KR); Jin Young Kim, Daejeon-shi (KR); Jong Shin Yoo, Daejeon-shi (KR); Young Mok Park, Daejeon-shi (KR); Seung Il Kim, Daejeon-shi (KR)

Correspondence Address: MERCHANT & GOULD PC, P.O. BOX 2903, MINNEAPOLIS, MN, 55402-0903, US

(73) Assignee: Korea Basic Science Institute, Daejeon-shi (KR)

(21) Appl. No.: 12/282,440

(22) PCT Filed: Feb. 23, 2007
(86) PCT No.: PCT/KR2007/000946

§ 371 Date: Sep. 10, 2008

(30) Foreign Application Priority Data
Feb. 22, 2007 (KR) 10- 2007-0017837

(51) Int. Cl. G06F 19/00; G01N 33/68

(52) U.S. Cl. 702/19

(57) ABSTRACT

The present invention relates to a method of analyzing protein modification. The method of invention for analyzing protein distribution and characteristics on one-dimensional gel provides the way to analyze proteins of samples on one-dimensional gel quantitatively and provides information on interactions among proteins and further can be effectively used for the development of a novel diagnostic and therapeutic method for a disease by screening a disease marker protein.

### TECHNICAL FIELD

The present invention relates to a method of analyzing protein modification which provides more specific information on proteome, in the proteomics research identifying proteins based on tandem mass spectrometry.

### BACKGROUND ART

Biological samples are largely composed of a variety of proteins. The series of separation methods such as one-dimensional SDS-PAGE or liquid chromatography separates proteins or peptides resulted from hydrolysis of the proteins included in those samples. And then the isolated proteins or peptides proceed to tandem mass spectrometry to give tandem mass spectra of peptides. Each amino acid sequence corresponding to each tandem mass spectrum can be screened from protein sequence database and further be identified by integrated analysis. For the screening of such protein or peptide sequences, softwares such as SEQUEST® (Eng et al., J. Am. Soc. Mass Spectrum. 5:976-989, 1994; Thermo Electron Corp., USA), Mascot (Perkins et al., Electrophoresis, 20:3551-3567, 1999; Matrix Science Ltd., USA, http://www.matrixscience.com/search_form_select.html), Sonar (Field, H. I. et al., Proteomics, 2:36-47, 2002; http://knxs.bms.umist.ac.uk/prowl/sonar/sonar_cntrl.html), X!Tandem (Craig et al., Bioinformatics, 20:1466-1467, 2004; Proteome Software Inc., USA), Phenyx, Peptide Prophet (Keller A., et al., Anal. Chem. 2002, 74, 5383-5392), Protein Prophet (Nesvizhskii A. I., et al., Anal. Chem. 2003, 75, 4646-4658), DTASelect (Tabb D. L., et al., Proteome Res. 2002, 1, 21-26) or OMSSA (Syka J E, et al., Proc Natl Acad Sci USA. 2004. Jun. 29, 101(26). 9528-33) can be used.

During the identification of a protein by screening the peptide sequence corresponding to tandem mass spectrum, the detection of the same proteins on different one-dimensional gel bands can happen in one of the cases indicates that the protein identification result was false positive or the identified protein is much abundant, or the protein modification is induced. However, there has been no method to distinguish these three possible cases by analyzing the experimental result.

To identify proteins separated from one-dimensional SDS-PAGE, each band of the one-dimensional gel is examined to find out corresponding protein sequences. If a protein is modified and thus exists in a sample in several different molecular weights, the protein can be detected on several bands of one-dimensional gel. So, investigation of each band position of one-dimensional gel leads to the quantitative analysis of modified proteins.

In previous patent publications, mass spectrums of peptides treated with different isotopes have been compared, with which protein mass analysis has been performed (US 2005/0233399). However, the method for mass analysis by isotope treatment was basically designed to analyze the mass of a protein which was equally modified but found in different samples, so it cannot be used for mass analysis of a protein that exists in different status in the same sample. The mass analysis with mass spectrums using a specific marker for a protein in a standard sample (US 2006/0078960) is also limited to the analysis of proteins especially when the amounts of proteins in a sample are similar to that of the standard sample, suggesting that this method is not preferred for the analysis of a protein in different status either. G. W. Park et al compared the results of identification of proteins in human serum and bacteria sample by tandem mass spectrometry with band positions of one-dimensional SDS-PAGE and confirmed the above results (G. W. Park, et al., Proteomics, 2006, 6, 1121-1132). However, at this time, only the band where the peptides of one protein are the most rich was selected for comparison. In most cases, modified proteins and non-modified proteins coexist. Q. R. Ahmad et al identified proteins of lymphoblastoid cells gathered in one-dimensional gel bands, among which 80% were identified as unmodified and 20% were modified proteins (Q. R. Ahmad, et al., Proteome Science, 2005, 3:6). However, this analysis was performed only with major populations and thus various proteins modified in different forms, which were minors though, were not included.

Therefore, the present inventors designed a method facilitating quantitative analysis of proteins in different samples by measuring proteins distributed in one-dimensional gel bands and also facilitating quantitative analysis of different proteins in one sample. As a result, according to this method, quantitative analysis of proteins in different concentrations identified in proteomics experiments can be possible without using the standard sample used for quantitative analysis of certain proteins. The present invention can provide precise, specific information on protein modification by analyzing different status and forms of a protein and further analyzes co-existence of different modified proteins and their original forms.

The present inventors designed a method for identifying proteins simultaneously found in multiple bands of one-dimensional gel by screening database to check errors and analyzing protein distribution thereon according to protein modification and then completed this invention by minimizing protein screening errors and giving the explanations on the protein modification.

### DISCLOSURE

### Technical Problem

It is an object of the present invention to provide a method of analyzing protein modification by analyzing tandem mass spectrums of one-dimensional gel and band positions in order to identify a protein efficiently.

### Technical Solution

Descriptions of Terms

Terms of the present invention are described as follows to increase understanding of this invention:

One-dimensional SDS-PAGE (sodium dodecyl sulphate-polyacrylamide gel electrophoresis) is a method of separating a protein by its molecular weight, which is the electrophoresis using polyacrylamide gel performed after regulating the ratio of electric charge to molecular weight of a protein using SDS (sodium dodecyl sulphate).

Tandem mass spectrometry is a method of analyzing mass of a protein by taking advantage of two different TOFs (time of flight), which are low speed TOF1 for parent ion separation and high speed TOF2 for fragment mass analysis.

Cluster indicates a group of peptides detected in consecutive bands, precisely if same peptides are detected in consecutive bands of one-dimensional gel, when a distribution map is made with those peptides identified by band positions, they are grouped in one and named cluster.

Island indicates the cluster of each protein. The strength of an island is determined by the sum of peptides identified as a corresponding protein in a cluster, the size of an island is determined by the width of a band and the location of an island indicates the central value of MWcorr (Mathematical Formula 2 below) calculated from each band.

Dispersion degree indicates the degree of protein dispersion determined by the relative ratio of peptides at the positions of representative bands of islands. In this invention, dispersion degree is indicated as I-score, which is calculated by the sum of Euclidean distances of islands from the island with the strongest strength (Mathematical Formula 1 below).

Molecular weight correlation (MWcorr) indicates the ratio of the theoretical molecular weight calculated with amino acid sequences of the corresponding protein to the experimental molecular weight converted from the one-dimensional electrophoresis moved positions (Mathematical Formula below).

### DISCLOSURE OF THE INVENTION

The present invention is described in detail.

To achieve the above object, the present invention provides the system of analyzing protein modification comprising the following means:

a) An interface for the reception of the information on tandem mass spectrums of peptides digested from each one-dimensional electrophoresis band loaded with the sample containing proteins;

b) A peptide identification method that is able to identify a peptide by comparing the tandem mass spectrum with protein sequence database;

c) A means making peptide dispersion map according to the numbers of peptides identified by the band position of one-dimensional electrophoresis;

d) A filtering means that eliminates the bands exhibiting smaller numbers of peptides compared with the highest numbers of peptides detected on one band by regarding the bands as noise;

e) A calculation means for peptide identification ratio that divides the number of peptides of each band by the total number of peptides excluding noises;

f) A clustering means, precisely when peptides are detected in consecutive bands these peptides are grouped as one cluster, and the band with the highest peptide rate of each cluster is selected as the representative band position and then each cluster is defined as an island;

g) A calculation means for island peptide rate which is obtained from the summation of peptide rate included in the island;

h) A calculation means for protein dispersion degree which calculates the dispersion related to the position and peptide rate of islands relative to the island exhibiting the highest peptide level; and

i) An output means that displays the dispersion degree according to the dispersion map of the peptides and proteins.

The present invention also provides the method of analyzing protein modification comprising the following steps:

1) Obtaining tandem mass spectrums using a mass spectrometer, in which the sample of protein mixture proceed to one-dimensional electrophoresis, each band is cut out, proteins are separated from the bands, the separated proteins are digested with a protease, and tandem mass spectrums of the peptides are obtained by a mass spectrometer;

2) Identifying the obtained peptides by comparing the tandem mass spectrums inputted through the interface connected to a mass spectrometer with protein sequence database;

3) Making distribution map with the number of peptides identified according to the band position;

4) Eliminating noise, in which bands exhibiting low amount peptides, which means the number of peptides does not meet the threshold ratio determined by the number of peptides of the band with highest density (the biggest peptide population), are eliminated as being considered as noises;

5) Calculating peptide ratio by dividing the number of peptides of each band by the sum of peptide numbers over the whole bands;

6) Determining each cluster as an island, in which peptides identified in consecutive bands are grouped as one cluster, and then the band with the highest peptide rate is selected as the representative band, and then each cluster is defined as an island;

7) Calculating peptide ratio in cluster as the total peptide ratio over the cluster; and

8) Calculating dispersion degree based on the position of each island and peptide ratio of each band, precisely the position of the island having the largest number of identified peptides among islands and peptide ratio therein are investigated.

Hereinafter, the present invention is described in detail.

In the protein analysis system, the interface of a) is preferably RSC-232C, parallel port, universal serial bus (USB), IEEE 1394, Bluetooth or Ethernet, but not always limited thereto.

In this analysis system, the protein sequence database of b) is preferably IPI_Human protein sequence database, UniprotKB/Swissprot database or NCBl_nr database, but not always limited thereto and each database can be downloaded at the following internet addresses. It is important to sort out wrong spectrums of peptides for the efficient protein identification. Thus, to increase the reliability, reverse sequence database can also be used together.

IPI: ftp://ftp.ebi.ac.uk/pub/databases/IPI/current/

UniprotKB/Swissprot: ftp://ftp.expasy.org/databases/uniprot/

NCBI_nr: ftp://ftp.ebi.ac.uk/pub/databases/

In the above analysis system, the certain ratio of d) is preferably 10% of the total number of peptides in the band showing the highest peptide population, buy not always limited thereto, and the dispersion degree can be calculated by I-score following the mathematical formula 1.

$${Iscore}_{j} = {\sum }_{i = 1}^{n} \sqrt{\frac{{( {x}_{P} - {x}_{i} )}^{2} + {( {y}_{P} - {y}_{i} )}^{2}}{1 + {( {y}_{P} - {y}_{i} )}^{2}}} \qquad [ Mathematical Formula 1 ]$$

j: jth protein among identified proteins.

(χp,yp): χp indicates the position of the island having the highest peptide rate of jth protein and yp indicates the peptide rate of this island. The position of an island is determined by the normalized value from 0 to 1.

(χi,yi): χi indicates the position of ith island of jth protein and yi indicates its peptide rate.

In the above analysis system, the output means of i) is preferably a monitor, a printer or a plotter, but not always limited thereto.

In the method of analyzing protein modification, one-dimensional electrophoresis of step 1) is preferably performed using SDS (sodium dodecyl sulphate) to regulate the rate of electric charge to molecular weight of a protein, followed by SDS-PAGE (sodium dodecyl sulphate-polyacrylamide gel electrophoresis) using polyacrylamide gel to separate the protein. The present inventors separated a protein from biological sample or protein mixture using SDS-PAGE, hydrolyzed thereof using trypsin and then identified the peptide by tandem mass spectrometry.

In the method of analyzing protein modification, the tandem mass spectrums obtained in step 1) are preferably analyzed by human protein database IPI_Human protein sequence database, UniprotKB/Swissprot database or NCBI_nr database, but not always limited thereto and those databases can be downloaded at the above addresses. To increase the reliability, reverse sequence database can be used together.

The sequence information is preferably in FASTA format but not always limited thereto and the general sequence screening software can be used for protein identification. The sequence screening software is preferably SEQUEST® (Eng et al., J. Am. Soc. Mass Spectrum. 5:976-989, 1994; Thermo Electron Corp., USA), Mascot (Perkins et al., Electrophoresis, 20:3551-3567, 1999; Matrix Science Ltd., USA, http://www.matrixscience.com/search_form_select.html), Sonar (Field, H. I. et al., Proteomics, 2:36-47, 2002; http://knxs.bms.umist.ac.uk/prowl/sonar/sonar_cntrl.html), X!Tandem (Craig et al., Bioinformatics, 20:1466-1467, 2004; Proteome Software Inc., USA), Phenyx, Peptide Prophet (Keller A., et al., Anal. Chem. 2002, 74, 5383-5392), Protein Prophet (Nesvizhskii A. I., et al., Anal. Chem. 2003, 75, 4646-4658), DTASelect (Tabb D. L., et al., Proteome Res. 2002, 1, 21-26) or OMSSA (Syka J E, et al., Proc Natl Acad Sci USA. 2004. Jun. 29, 101(26). 9528-33), but not always limited thereto.

In the method of analyzing protein modification, the interface of step 2) is preferably RSC-232C, parallel port, universal serial bus (USB), IEEE 1394, Bluetooth or Ethernet, but not always limited thereto.

In the method of analyzing protein modification, the distribution map of step 3) is made as follows; among identified bands, the band with highest identified peptide population is selected and any band determined to contain less than 10% peptides compared with the highest peptide band is considered as noise and thus eliminated (step 4). Peptide identification rate is calculated by dividing the number of peptides identified in each band by the total number of peptides (step 5) and if peptides are identified in consecutive bands, they are grouped in one and named as cluster. The band exhibiting the highest peptide rate in each cluster is determined to be the representative band position and each cluster is indicated as ‘island’ (step 6). The islands can simplify the complicated protein patterns of one-dimensional gel (see FIG. 2).

The dispersion degree of step 8) represents protein dispersion based on the representative band positions of islands originated from same protein and peptide rate, which is calculated by I-score (IScore; see FIG. 3) of the above mathematical formula 1. This dispersion degree facilitates quantitative analysis of modified proteins. If a protein has only one island, I-score will be 0. However, proteins digested or modified by any enzyme before proceeding to one-dimensional gel electrophoresis have multiple numbers of islands and thus the value of I-score increases. Thus, if I-score of a protein is low but the size of an island is big, this protein is expected to be highly abundant. I-score increases when a protein is dispersed in several bands far from each other, while I-score is 0 when a protein is crowded in one place. Therefore, I-score can be effectively used for quantitative analysis of protein dispersion. In general most proteins have low I-scores and smaller islands, indicating that they are well-localized in the 1D-SDS gel.

The method of analyzing protein modification of the present invention can further contain the following step:

9) Comparing the modifications of a whole proteome with other samples based on island dispersion.

The information on protein modification obtained from the above analysis (see FIG. 1) can be used as basic data for screening the genome information, interaction of proteins and metabolism information in biological samples or protein mixture.

The method of analyzing protein modification of the present invention can further contain the following steps:

9) Comparing island distribution of each protein with protein modification in the corresponding protein;

10) Analyzing protein distribution by applying the dispersion degree to different species or different samples; and

11) Comparing and determining protein modification patterns of different species or different samples by arranging and diagramming protein distribution according to the size of dispersion degree based on the calculated molecular weight correlation (MWcorr) values to outline the characteristics of a whole proteome.

In step 9), if the distribution of islands is bigger than the molecular weight calculated based on the amino acid sequence, it can be expected that N-glycosylation is induced through the informed protein modification (see FIG. 4).

The informed protein modification in step 9) is preferably analyzed by protein database such as Swiss-Prot database, NCBI_nr database or UniProt database and protein modification predicting software such as SignalP or GlycoSuite, but not always limited thereto.

In step 11), MWcorr (Molecular Weight Correlation) is calculated by dividing log(MWexp) by log(MWcal), which means logarithmic ratio of the molecular weight obtained from amino acid sequence (MWcal) and the value converted from band position of one-dimensional gel (MWexp). And the MWcorr is defined as the following mathematical formula 2. If MWcorr is 1, the molecular weight calculated from one-dimensional gel band position is the same as the molecular weight calculated with amino acid sequence. If MWcorr is less than 1, the molecular weight calculated from one-dimensional gel band position is lower than that resulted from the calculation with amino acid sequence. On the contrary, if MWcorr is higher than 1, the molecular weight obtained from one-dimensional gel band position is higher than that resulted from the calculation with amino acid sequence. When MWcorr is higher than 1, protein modification is induced by binding with high molecular weight proteins in many cases, while when MWcorr is lower than 1, proteins are cut off and thus reduced in their molecular weights.

The distribution maps were made with islands from the proteins with small I-score to the proteins with big I-score with various samples. In the case of human serum samples, proteins were scattered in the regions having MWcorr more than or less than 1 (see FIG. 5) and in the case of human brain tissue samples, proteins were crowded in the region having MWcorr more than 1 (see FIG. 6). In the case of Pseudomonas putida KT2440 bacteria, proteins were crowded in the region having MWcorr to be 1 (see FIG. 7).

The islands and I-score can be efficient to give simple explanations on the complicated protein modifications. Therefore, along with MWcorr, the maps of identified proteins (see FIG. 4-FIG. 7) from various samples can contribute to many interesting biological studies including alternative splicing, endoproteolytic process or posttranslational modification (PTM).

$$M {W}_{corr} = \frac{log M {W}_{exp}}{log M {W}_{cal}} \qquad [ Mathematical Formula 2 ]$$

MWcal; molecular weight of a protein calculated from amino acid sequence.

MWexp; molecular weight of a protein calculated with one-dimensional gel band position.

### DESCRIPTION OF DRAWINGS

The application of the preferred embodiments of the present invention is best understood with reference to the accompanying drawings, wherein:

FIG. 1 is a diagram illustrating the processes of separating proteins from biological samples or protein mixture by one-dimensional SDS-PAGE electrophoresis and analyzing protein modification using tandem mass spectrometry.

FIG. 2 is a diagram illustrating the process of calculating major band positions of a protein.

FIG. 3 is a diagram illustrating the method for determining relative distribution of I-score of peptides identified as protein j.
n: number of islands;
xp: position of the island where peptides are identified most;
yp: peptide rate of the island where peptides are identified most;
xi: position of ith island; and
yi: rate of the peptide identified as protein j of the ith island.

FIG. 4 is a diagram illustrating protein sequences corresponding to the band position of a glycoprotein and the band position of modified protein with deletion of a part of the corresponding protein.

FIG. 5 is a diagram illustrating band positions and quantitative distribution of proteins of human serum samples classified by the size of I-score. Proteins are arranged from left to right according to the size of I-score. The circles in vertical direction indicate the distribution of islands where one protein is identified. We colored the circles as red for abundant peptides, blue for the low abundant peptides.

FIG. 6 is a diagram illustrating band positions and quantitative distribution of proteins of human brain tissue samples classified by the size of I-score. Proteins are arranged from left to right according to the size of I-score. The circles in vertical direction indicate the distribution of islands where one protein is identified. We colored the circles as red for abundant peptides, blue for the low abundant peptides.

FIG. 7 is a diagram illustrating island positions and quantitative distribution of proteins of Pseudomonas putida KT2440 bacteria classified by the size of I-score. Proteins are arranged from left to right according to the size of I-score. The circles in vertical direction indicate the distribution of islands where one protein is identified.

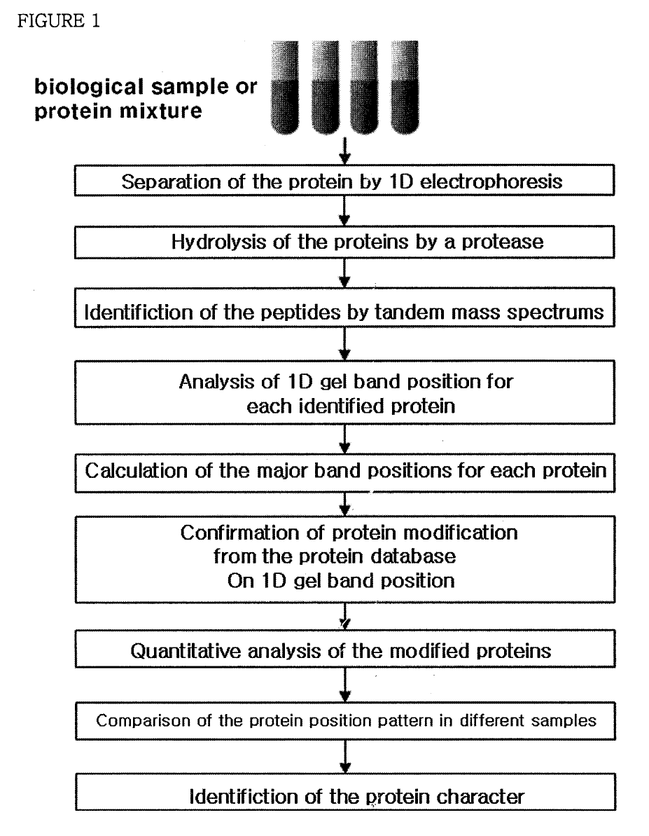

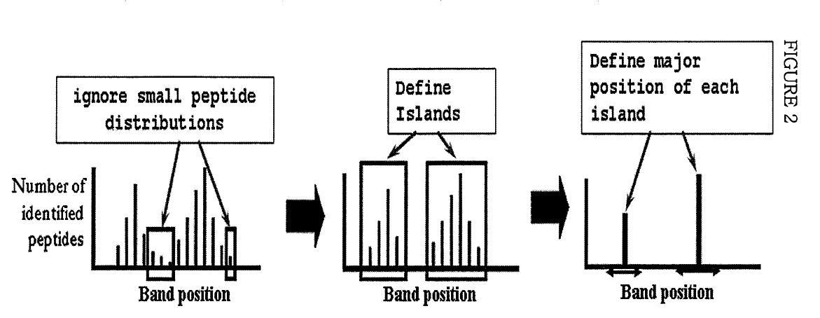

### BEST MODE

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Analysis of Protein Modification in Human Serum Samples

<1-1> One-Dimensional SDS-PAGE with Human Serum Samples

Major abundant proteins in human serum samples were eliminated by using MAR affinity column [MAR column (4.6×50 mm2), Agilent]. The eliminated proteins were albumin, immunoglobullins (Igs) A and G, haptoglobin, transferrin and antitrypsin. The proteins with the elimination of those 6 proteins were separated by one-dimensional SDS-PAGE using 12% acrylamide gel. The size of one lane of one-dimensional gel was 18 cm×1 cm×0.1 cm. 100 μg of human blood sample was loaded on gel, followed by electrophoresis at 100 volt for about 4 hours. Upon completion of electrophoresis, protein bands were detected by staining with CBB (Coomassie brilliant blue). 70 stained bands were extracted.

<1-2> Separation of Peptides and Obtainment of Tandem Mass Spectrums from One-Dimensional Gel

Peptides were extracted from each band of one-dimensional gel obtained by one-dimensional electrophoresis (one-dimensional SDS-PAGE) of Example <1-1> by multidimensional protein identification technology (MudPIT) as described by Pieper et al (Pieper, R., et al., Proteomics, 3: 422-432, 2003).

70 bands of one-dimensional gel were cut and hydrolyzed with trypsin, and the resultant peptide mixture was inputted into 250 μm tubing (UK) filled with C18, SCX cation exchange materials (Whatman column, UK) by 2-3 cm. Tandem mass spectrums were obtained by using a mass spectrometer (LTQ-FT, Thermo Electron Corp., CA).

The obtained tandem mass spectrums were analyzed by the IPI_Human protein sequence database version 3.06 (ftp://ftp.ebi.ac.uk/pub/databases/IPI/current/) downloaded from EBI (UK). To identify proteins with high efficiency, it is important to sort out wrong spectrums at peptide level. Thus, the present inventors used reverse sequence database to calculate the ratio of false positive identifications and identified peptides at the error rate of 1%. From the peptides filtered by molecular weight distribution (−9.55 ppm≦ΔM≦15.76 ppm), proteins were identified with high accuracy. Protein identification was performed with the protein identification software (TurboSEQEST®, Thermo Electron Corp., USA).

<1-3> Analysis of Protein Modification

Among bands of one-dimensional gel, those bands having less than 10% of the peptides that were identified as the corresponding proteins in the spectrums were eliminated. And then consecutive bands containing identified peptides were grouped as a cluster. Each cluster was defined as an island. The strength of an island is determined by the sum of the peptides identified as the corresponding protein and the size of an island is defined by the width of a band. The position of an island is determined by the central value of MWcorr (Mathematical Formula 2) calculated from each band.

The distance from the island exhibiting the higher intensity than the other islands was calculated (Euclidean distance), resulting in I-score (Mathematical Formula 1).

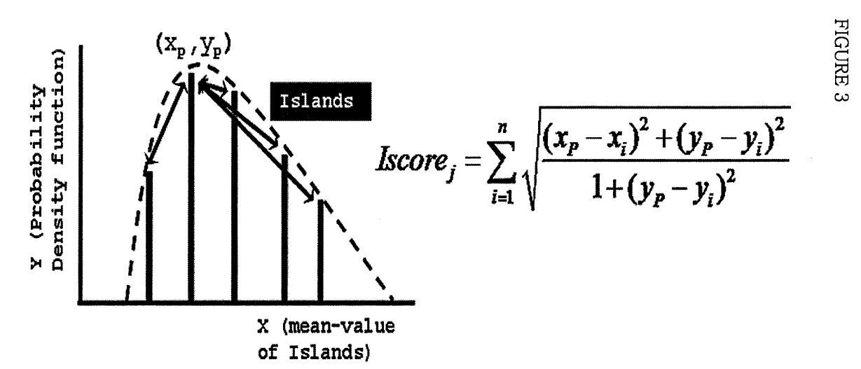

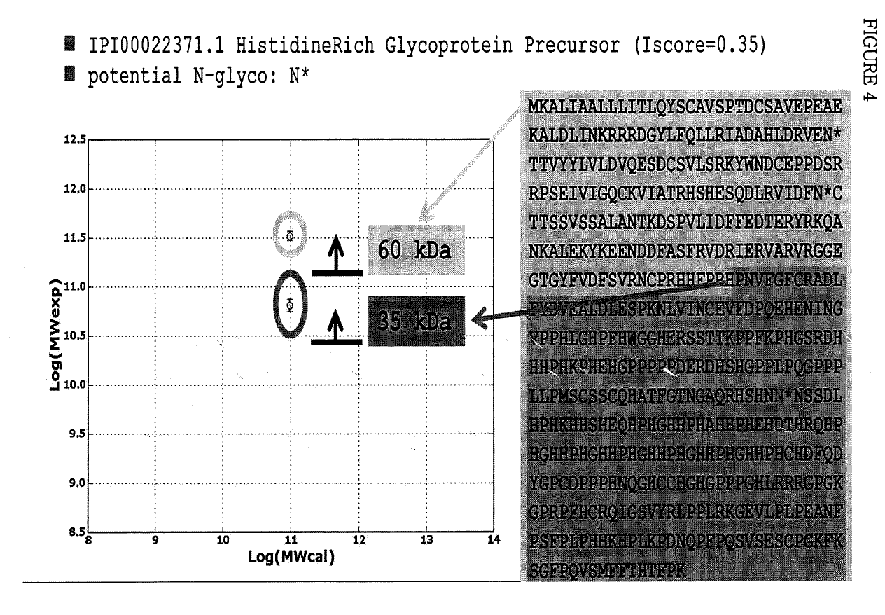

Among proteins identified from IPI_Human database of Example <1-2>, IIPI00022371.1 Histidine Rich Glycoprotein Precursor had two islands, confirmed from the island detection (FIG. 2), and had I-score of 0.35 (FIG. 4) calculated from the above Mathematical Formula 1 (FIG. 3). Any similar sequences to the corresponding protein had been screened from NCBI_nr protein database. And as a result, lower molecular weight (49 kDa) island among two islands was correspondent to the molecular weight of a fraction cut off in the middle of the whole amino acid sequence. The proteins screened from NCBI_nr were “gi|2280514|” and “gi|2280514|”. The positions of the islands (MWcorr=0.98 and MWcorr=1.05) exhibited rather higher molecular weights (49 kDa and 99 kDa) than the predicted molecular weight calculated from the amino acid sequence, which were 35,366 Da and 59,540 Da, which was conjectured to be occurred because the N-glycosylation increased molecular weights, and confirmed by Swiss-Prot data. The results also indicates that posttranslational modification (PTM) was induced.

### Example 2

### Analysis of Protein Modifications in Different Species

Protein identification and island analysis were performed with human brain tissues and Pseudomonas putida KT2440 bacteria by the same manner as described in Example 1, the experiment with human serum samples. But in this example, human brain tissue samples proceeded to one-dimensional electrophoresis and 40 bands were separated from one-dimensional gel. Each band was treated with trypsin and then peptide identification was performed by using fused-silica tubing (Phenomenex, USA) filled with 10 cm of Aqua 5μ C18 with a mass spectrometer (LT LTQ/MS, Linear Ion Trap Mass Spectrometer, Thermo Electron Corp., USA). 42 bands were extracted from the bacteria samples and the peptide mixture hydrolyzed with trypsin was inputted in 250 μm tubing (UK) filled with 2-3 cm of SCX cation exchange materials (Whatman column, UK) and then tandem mass spectrums were obtained by a mass spectrometer (LT LTQ/MS, Linear Ion Trap Mass Spectrometer, Thermo Electron Corp., USA).

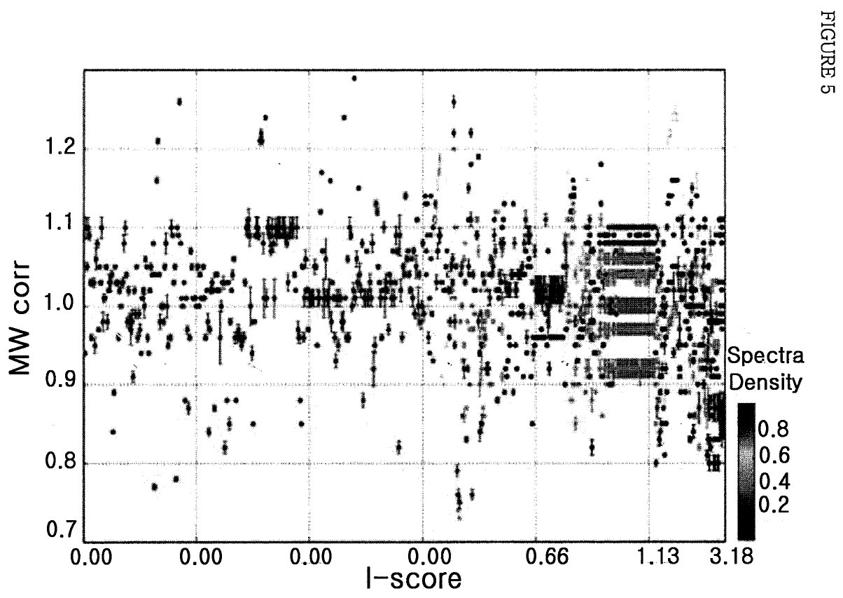

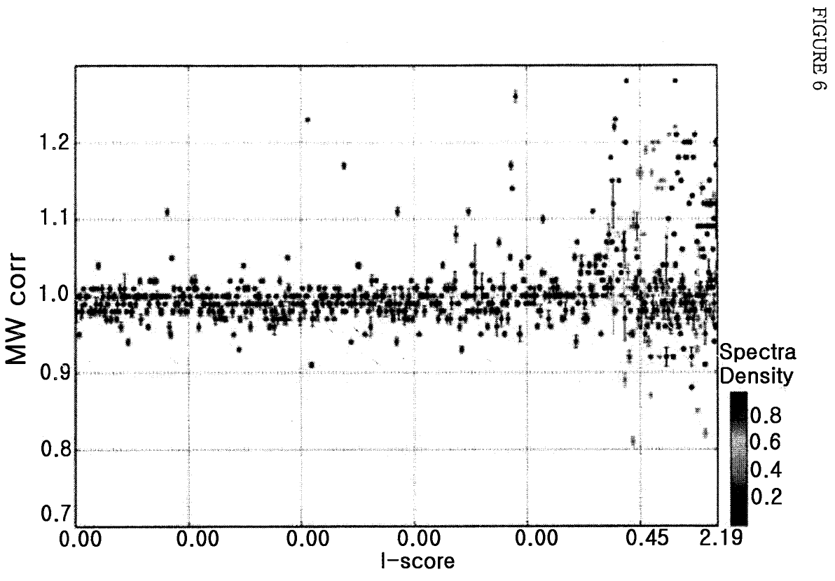

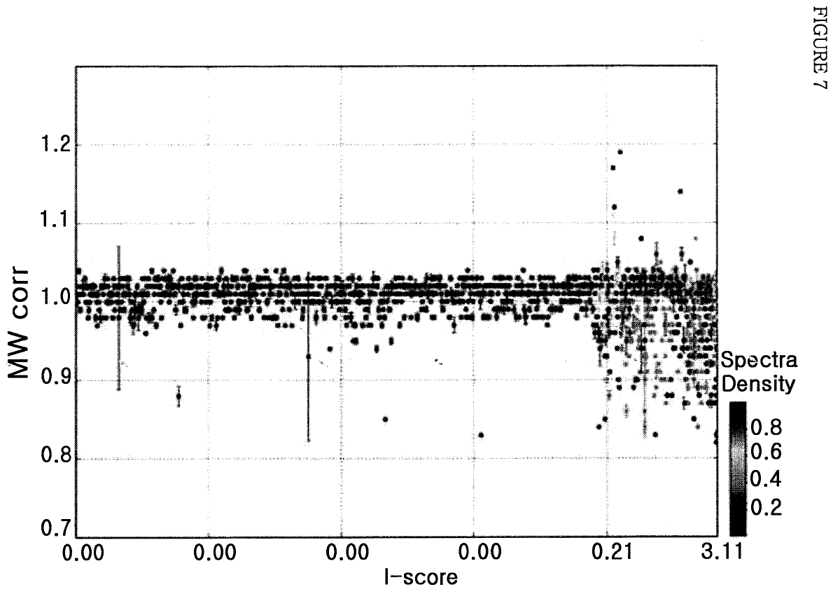

Islands of proteins identified from human serums and brain tissues and Pseudomonas putida KT420 bacteria samples were analyzed and I-scores were obtained. MWcorr (Molecular Weight Correlation) was measured by the above Mathematical Formula 2. As a result, 482, 579 and 965 proteins were identified respectively from human serums, human brain tissues and bacteria. In the case of human serum samples, proteins were dispersed in the regions with MWcorr value of higher than 1 or lower than 1 (FIG. 5). In the case of human brain tissue samples, proteins were specifically crowded in the region with MWcorr value of higher than 1 (FIG. 6). In the case of bacteria samples, proteins with lower I-score were gathered in the region with MWcorr value of 1 but those with higher I-score were proved to be fractionated (FIG. 7).

### INDUSTRIAL APPLICABILITY

As explained hereinbefore, the method of analyzing protein modification by using tandem mass spectrum data and one-dimensional gel band positions is clearly advanced from the conventional method simply identifying proteins and detecting the positions of representative proteins on one-dimensional gel. So, the method of the invention provides the way to analyze distribution on one-dimensional gel quantitatively and provides information on modifications of proteins in each sample. Therefore, the method of the invention can be effectively used for investigation of interaction among proteins and protein metabolism pathway and screening for a disease marker.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A system of analyzing protein modification, which comprises:
a) An interface for the reception of the information on tandem mass spectrums of digested peptides from each one-dimensional electrophoresis band loaded with samples containing proteins;
b) A means for peptide identification that is able to identify a peptide by comparing the tandem mass spectrum with protein sequence database;
c) A means making peptide dispersion map according to the numbers of peptides identified by the band positions of one-dimensional electrophoresis;
d) A filtering means that eliminates the bands exhibiting small number of peptides under the threshold ratio compared with the highest numbers of peptide detected on the band having the majority by recognizing the bands as noises;
e) A calculation means for peptide identification ratio that divides the number of peptides of each band by the total number of peptides excluding noises;
f) A clustering means, precisely when peptides are detected in consecutive bands these peptides are grouped as one cluster, and the band with the highest peptide rate of each cluster is selected as the representative band position and then each cluster is defined as an island;
g) A calculation means for island peptide rate;
h) A calculation means for protein dispersion degree, precisely among islands, those exhibiting the highest peptide level are selected and based on the positions and peptide rates of such identified islands, the position of each island and dispersion degrees of peptides are calculated; and
i) An output means that displays the dispersion degree according to the dispersion map of the peptides and proteins.

2. The system of analyzing protein modification according to claim 1, wherein the interface of a) is RSC-232C, parallel port, universal serial bus (USB), IEEE 1394, Bluetooth or Ethernet.

3. The system of analyzing protein modification according to claim 1, wherein the protein sequence database of b) is IPI_Human protein sequence database, UniprotKB/Swissprot database, NCBI_nr database and/or their reverse sequence database.

4. The system of analyzing protein modification according to claim 1, wherein the threshold ratio of d) is 10% of the total number of peptides in the band showing the highest peptide population

5. The system of analyzing protein modification according to claim 1, wherein the dispersion degree of h) is calculated by the following mathematical Formula 1.
$${Iscore}_{j} = {\sum }_{i = 1}^{n} \sqrt{\frac{{( {x}_{P} - {x}_{i} )}^{2} + {( {y}_{P} - {y}_{i} )}^{2}}{1 + {( {y}_{P} - {y}_{i} )}^{2}}} \qquad < Mathematical Formula 1 >$$
j: jth protein among identified proteins.
(χp,yp): χp indicates the position of the island having the highest peptide rate of jth protein and yp indicates the peptide rate of the said island. The position of an island is determined by the normalized value from 0 to 1.
(χi,yi): χi indicates the position of ith island of jth protein and yi indicates peptide rate.

6. The system of analyzing protein modification according to claim 1, wherein the output means of i) is a monitor, a printer or a plotter.

7. A method of analyzing protein modification comprising the following steps:
1) Obtaining tandem mass spectrums using a mass spectrometer, in which protein containing samples proceed to one-dimensional electrophoresis, each band is cut out, proteins are extracted from the bands, the separated proteins are digested with a protease, and tandem mass spectrums of the peptides are obtained by a mass spectrometer;
2) Identifying the obtained peptides by comparing the tandem mass spectrums inputted through the interface connected with a mass spectrometer with protein sequence database;
3) Making distribution map with the number of peptides identified according to the band position;
4) Eliminating noise, in which bands exhibiting smaller amount of peptides, which means the number of peptides does not meet the threshold ratio determined by considering the number of peptides of the band with highest density (the biggest peptide population), are eliminated as being considered as noise;
5) Calculating peptide identification ratio by dividing the number of peptides of each band by the sum of peptide numbers;
6) Determining each cluster as an island, in which peptides identified in consecutive bands are grouped as one cluster, and then the band with the highest peptide rate is selected as the representative band, and then each cluster is defined as an island;
7) Calculating peptide ratio in cluster; and
8) Calculating dispersion degree based on the position of each island and peptide ratio of each band, precisely the position of the island having the largest number of identified peptides among islands and peptide ratio therein are investigated.

8. The method of analyzing protein modification according to claim 7, wherein the step of 9) Comparing the modifications of a whole proteome in different samples based on island distribution is additionally included.

9. The method of analyzing protein modification according to claim 7, wherein the one-dimensional electrophoresis of step 1) is SDS-PAGE (sodium dodecyl sulphate-polyacrylamide gel electrophoresis).

10. The method of analyzing protein modification according to claim 7, wherein the interface of step 2) is RSC-232C, parallel port, universal serial bus (USB), IEEE 1394, Bluetooth or Ethernet.

11. The method of analyzing protein modification according to claim 7, wherein the protein sequence database of step 2) is IPI_Human protein sequence database, UniprotKB/Swissprot database, NCBI_nr database and/or their reverse sequence database.

12. The method of analyzing protein modification according to claim 11, wherein the database sequence information is in FASTA format.

13. The method of analyzing protein modification according to claim 7, wherein the protein identification of step 2) is performed by one of the protein identification software selected from a group consisting of SEQUEST® Mascot, Sonar, X!Tandem, Phenyx, PeptideProphet, Protein Prophet, DTASelect and OMSSA.

14. The method of analyzing protein modification according to claim 7, wherein the dispersion degree of step 8) is calculated by the following Mathematical Formula 1.
$${Iscore}_{j} = {\sum }_{i = 1}^{n} \sqrt{\frac{{( {x}_{P} - {x}_{i} )}^{2} + {( {y}_{P} - {y}_{i} )}^{2}}{1 + {( {y}_{P} - {y}_{i} )}^{2}}} \qquad < Mathematical Formula 1 >$$
j: jth protein among identified proteins.
(χp,yp): χp indicates the position of the island having the highest peptide rate of jth protein and yp indicates the peptide rate of the said island. The position of an island is determined by the normalized value from 0 to 1.
(χi,yi): χi indicates the position of ith island of jth protein and yi indicates peptide rate.

15. The method of analyzing protein modification according to claim 7, wherein the following steps are additionally included:
9) Comparing island distribution of each protein with protein modifications in the corresponding proteins;
10) Analyzing protein distribution by applying the dispersion degree to different species or different samples; and
11) Comparing and determining protein modification patterns of different species or different samples by arranging and diagramming protein distribution according to the size of dispersion degree based on the calculated molecular weight correlation (MWcorr) values to outline the characteristics of the whole protein.

16. The method of analyzing protein modification according to claim 15, wherein the known information on protein modification of step 9) is provided by protein sequence database or the result of the analysis performed by using a protein modification predicting software.

17. The method of analyzing protein modification according to claim 16, wherein the protein sequence database is Swiss-Prot database, NCBI_nr database or UniProt database.

18. The method of analyzing protein modification according to claim 16, wherein the protein modification predicting software is SignalP or GlycoSuite.

19. The method of analyzing protein modification according to claim 15, wherein the MWcorr of step 11) is calculated by the following Mathematical Formula 2.
$$M {W}_{corr} = \frac{log M {W}_{exp}}{log M {W}_{cal}} \qquad < Mathematical Formula 2 >$$
MWcal: molecular weight of a protein calculated from amino acid sequence;
MWexp: molecular weight of a protein calculated with one-dimensional gel band position.


SEQUENCE LISTING (1 sequence)

SEQ ID NO: 1 - protein, 525 aa, Homo sapiens
MKALIAALILITLQYSCAVSPTDCSAVEPEAEKALDLINKRRRDGYLFQLIRIADAHLDRVENTTVYYLVLDVQESDCSVISRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFRRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK